(19) United States
(12) Patent Application Publication
(10) Pub. No.: US 2026/0284271 A1
(43) Pub. Date: Sep. 24, 2026

(54) HYALURONIC ACID COMPOSITIONS WITH LOW EXTENTS OF ESTER LINKAGES AND METHODS OF USE THEREOF

(71) Applicant: Prohibix LLC, Philadelphia, PA (US)

(72) Inventor: Brendan Purcell, Boulder, CO (US)

(73) Assignee: Prohibix LLC, Philadelphia, PA (US)

(21) Appl. No.: 19/475,258

(22) PCT Filed: Apr. 12, 2024
(86) PCT No.: PCT/US2024/024399

§ 371 Date: Oct. 14, 2025

Related U.S. Application Data
(60) Provisional application No. 63/496,226, filed on Apr. 14, 2023.

(51) Int. Cl. A61L 27/52; A61L 27/20; A61L 27/54

(52) U.S. Cl. CPC A61L 27/52; A61L 27/20; A61L 27/54; A61L 2400/06; A61L 2400/10; A61L 2430/38

(57) ABSTRACT

Compositions comprised of hyaluronic acid with low extents of ester linkages are disclosed. The compositions may enable longer half-lives of hyaluronic acid delivered in tissues throughout the body to prolong mechanical effects through controlled swelling and solubilization of hyaluronic acid as ester crosslinks between hyaluronic acid polymers hydrolyze. The compositions may also enable longer half-lives of medicaments or drugs in tissues throughout the body as ester linkages between the medicaments or drugs and hyaluronic acid polymers or networks hydrolyze. Compositions may include acrylate modified hyaluronic acid with low degrees of acrylate modification in order to preserve the physical properties of hyaluronic acid in the body.

### CROSS REFERENCE TO RELATED APPLICATIONS

This application is a national phase entry of international Application No. PCT/US2024/024399, filed Apr. 12, 2024, which claims the benefit of U.S. provisional application No. 63/496,226, filed Apr. 14, 2023, titled HYALURONIC ACID COMPOSITIONS WITH LOW EXTENTS OF ESTER LINKAGES AND METHODS OF USE THEREOF, the contents of which are incorporated herein by reference as if fully set forth.

### GOVERNMENT SUPPORT STATEMENT

This invention was made with government support under grant number R43HL140645 awarded by the National Institutes of Health National Heart, Lung, and Blood Institute and R43AR079965 awarded by the National Institutes of Arthritis and Musculoskeletal and Skin Diseases. The government has certain rights in the invention.

### FIELD

This relates to compositions of hyaluronic acid for local administration in the body.

### BACKGROUND

Hyaluronic acid (HA) is a linear glycosaminoglycan composed of repeating D-glucuronic acid and N-acetyl-D-glucosamine disaccharides. It is a hydrophilic, water-swollen polymer, and is abundant in the extracellular matrix of tissues throughout the body. HA swelling has important mechanical and biological roles in regulating tissue hydration and mechanics, tissue formation, wound healing, and inflammation. HA polymers can be found in the body at molecular weights ranging from 5,000 daltons (Da) to 20,000,000 Da. HA dissolved in aqueous buffers form shear-thinning solutions with viscoelastic properties that are directly proportional to HA concentration and molecular weight. See Snetkov, P., et al., Hyaluronic Acid: The Influence of Molecular Weight on Structural, Physical, Physico-Chemical, and Degradable Properties of Biopolymer. Polymers (Basel), 2020. 12 (8), which is incorporated herein by reference as if fully set forth.

Crosslinks can be introduced between dissolved HA polymers at low extents of crosslinking to increase effective molecular weight or at higher extents of crosslinking to form hydrogels for increased stability in the body. See Burdick, J. A. and G. D. Prestwich, Hyaluronic acid hydrogels for biomedical applications. Adv Mater, 2011. 23 (12): p. H41-56, which is incorporated herein by reference as if fully set forth. HA hydrogels are typically fabricated by first dissolving HA or chemically modified HA in aqueous buffer and then initiating crosslinking through the addition of crosslinking molecules, chemical initiators or through mixing complimentary chemically modified HA. The crosslinked hydrogels swell when placed in aqueous buffer, and hydrogel swelling is inversely proportional to crosslink density. In addition, hydrogel mechanics and stability are directly proportional to crosslink density. The hydrophilic nature of HA is important for its mechanic roles in tissues throughout the body, but also limits the concentration of HA during the crosslinking reaction and, therefore, high extents of chemical modification are typically required to form an insoluble material.

HA hydrogels have been used as drug delivery vehicles by encapsulating the drug inside the hydrogel. The water swollen HA network acts to slow the diffusion of encapsulated molecules from the gel. Crosslink density and HA-molecule affinity can be tuned to further slow molecule release. See E. Tous et al., Injectable acellular hydrogels for cardiac repair. J Cardiovasc Transl Res, 2011. Oct; 4 (5): 528-42.

### SUMMARY

In an aspect, the invention relates to a composition comprising hyaluronic acid conjugated to medicaments and drugs through hydrolytically degradable bonds.

In an aspect, the invention relates to a composition comprising hyaluronic acid conjugated to medicaments and drugs through ester bonds.

In an aspect, the invention relates to a composition comprising hyaluronic acid linked to medicaments and drugs through hydrolytically degradable bonds.

In an aspect, the invention relates to a composition comprising hyaluronic acid linked to medicaments and drugs through ester bonds.

In an aspect, the invention relates to a composition comprising acrylate modified hyaluronic acid linked to medicaments and drugs.

In an aspect, the invention relates to a composition comprising acrylate modified hyaluronic acid with low extents of chemical modification linked to medicaments and drugs.

In an aspect, the invention relates to a composition comprising acrylate modified hyaluronic acid with less than 5 percent chemical modification linked to medicaments and drugs.

In an aspect, the invention relates to a composition comprising acrylate modified hyaluronic acid with less than 2 percent chemical modification linked to medicaments and drugs.

In aspect, the invention relates to a composition comprising a hydrolytically degradable network linked to medicaments or drugs through reaction to crosslinking groups.

In aspect, the invention relates to a composition comprising a hydrolytically degradable polymer network linked to medicaments or drugs through reaction to crosslinking groups.

In an aspect, the invention relates to a composition comprising acrylate modified hyaluronic acid with low extents of crosslinking.

In an aspect, the invention relates to a composition comprising acrylate modified hyaluronic acid with less than 5 percent crosslinking.

In an aspect, the invention relates to a composition comprising acrylate modified hyaluronic acid with less than 2 percent crosslinking.

In an aspect, the invention relates to a composition comprising a hyaluronic acid hydrogel with low extents of hydrolytically degradable crosslinking.

In an aspect, the invention relates to a composition comprising a hyaluronic acid hydrogel with low extents of ester crosslinking.

In an aspect, the invention relates to a composition comprising a hyaluronic acid hydrogel with low extents of disulfide crosslinking.

In an aspect, the invention relates to a composition comprising a hydrogel of hyaluronic acid with low extents of ester crosslinking and molecules linked to hydrogel through ester bonds.

In an aspect, the invention relates to a composition comprising a hydrogel of hyaluronic acid with low extents of ester crosslinking and medicaments and drugs linked to hydrogel through ester bonds.

In an aspect, the invention relates to a method of making a hydrogel of hyaluronic acid comprising modifying hyaluronic acid with low concentrations of acrylate groups and mixing with a multifunctional thiol to facilitate crosslinking.

In an aspect, the invention relates to a method of treatment comprising injecting into a body of a subject a composition comprising a crosslinked and acrylate modified hyaluronic acid, or the product of reacting an acrylate modified hyaluronic acid with a multifunctional thiol crosslinker.

In an aspect, the invention relates to a composition comprising hyaluronic acid crosslinked at low extents of ester crosslinking to form soft materials that retain the structure and properties of hyaluronic acid and have extended duration in the body.

In an aspect, medicaments or drugs are linked to acrylate modified and crosslinked hyaluronic acid to extend their duration in the body.

In an aspect, medicaments and drugs comprise the multifunctional crosslinker for acrylate modified hyaluronic acid.

In an aspect, the invention relates to hyaluronic acid microparticles that hydrate and swell over time as crosslinks hydrolyze to provide long-lasting mechanical effects in the body.

In an aspect, the invention relates to hyaluronic acid microparticles with linked medicaments or drugs that hydrate and swell over time as crosslinks hydrolyze to release medicaments or drugs.

In an aspect, the invention relates to a method of forming crosslinked hyaluronic acid hydrogels by mixing hyaluronic acid modified with acrylate groups at low extents of chemical modification with multifunctional thiol crosslinkers and incubating for extended periods of time.

In an aspect, the invention relates to a method of forming crosslinked hyaluronic acid hydrogels with linked molecules by mixing hyaluronic acid modified with acrylate groups at low extents of chemical modification with multifunctional thiol crosslinkers and incubating for extended periods of time and then swelling crosslinked network with medicaments and drugs to link to excess crosslinking groups.

In an aspect the medicaments and drugs are chemically modified to link to excess crosslinking groups.

In an aspect the invention relates to a use of a composition comprising hydrogel microparticles of acrylate modified hyaluronic acid, or the product of reacting acrylate modified hyaluronic acid with a multifunctional thiol crosslinker reconstituted in aqueous buffer for treating inflammation, treating osteoarthritis, treating pain, treating ocular disease, treating vaginal atrophy, treating cardiovascular disease, treating disc degeneration, treating diabetic ulcers, filling tissue volume, stimulating tissue regeneration, reducing the appearance of wrinkles, treating pain, providing hydration to tissues, reducing inflammation following surgery, improving tissue healing following surgery or a traumatic injury.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1A shows acrylate modified HA fabricated by reaction with acrylic anhydride in water pH 7.0-9.0. FIG. 1B shows the compressive moduli of hydrogels comprised of acrylate modified hyaluronic acid crosslinked with dithiothreitol (Mean+/−SD, n=3). FIG. 1C shows the swelling ratios of acrylate modified hyaluronic acid crosslinked with dithiothreitol following incubation in PBS at 37C.

FIG. 2A shows a schematic of the solubilization of ester crosslinked hyaluronic acid hydrogel microparticles over time. FIG. 2B shows rheological properties of solubilizing hyaluronic acid microparticles in a transwell incubation against phosphate buffered saline, pH 7.4, 37° C. (TA Instruments HR2, 20 mm cone and plate, 1 degree cone, 28 micron gap. Mean+/−SD, n=2 per timepoint).

FIG. 3 shows the total radiant efficiency of a cyanine 7.5 signal in the knee of rats following the intra-articular injection of cyanine 7.5 labeled hyaluronic acid hydrogel microparticle injection (Mean+/−SD, n=6 knees per timepoint, IVIS Lumina, 780/845 nm ex/em). The hyaluronic acid hydrogel microparticles were comprised of acrylate modified hyaluronic acid crosslinked with dithiothreitol. The hyaluronic acid was labeled with cyanine 7.5-amine through amide bond formation with carboxylic acid groups on hyaluronic acid prior to acrylate modification and crosslinking. The rheological properties of the particles are shown in Table 1.

FIG. 4 shows the total radiant efficiency of a cyanine 7.5 signal in the knee of rats following the intra-articular injection of ester crosslinked hyaluronic acid microparticles with cyanine 7.5 maleimide linked to excess thiol crosslinking groups. (Mean+/−SD, n=6 knees per timepoint, IVIS Lumina, 780/845 nm ex/em). The acrylate modified hyaluronic acid was crosslinked at 1.75 percent extent of crosslinking prior to swelling in cyanine 7.5 maleimide solution and administration via intra-articular injection (microparticle-Cy7.5). The same solution of cyanine 7.5 maleimide used for microparticle swelling and linking was administered at the same volume via intra-articular injection as a control (Cy7.5 maleimide).

### DETAILED DESCRIPTION

Certain terminology is used in the following description for convenience only and is not limiting. The words “right,” “left,” “top,” and “bottom” designate directions in the drawings to which reference is made.

“Network” is a collection of polymers in which all polymers are interconnected with each other through physical or chemical crosslinks.

“Hydrogel” is a water swollen network of polymers.

“Crosslink” refers to covalent or non-covalent bonds formed between polymers.

“Crosslinker” refers to molecules that form bonds between polymers. Crosslinkers can react with a polymer to form a crosslinking bond or be chemically modified onto the polymer prior to reacting to other polymers.

“Linked” refers to a molecule bound to a polymer through reaction to a chemical modification on the polymer or to a hydrogel polymer network through reaction to one or more crosslinking groups on the polymer or crosslinker.

“Chemical linker” or “linkage” is the chemistry between linked molecule and polymer.

“Extent of linking” refers to the percent of repeat units of a polymer bound to a linked molecule. For HA, the repeat unit is the disaccharide composed of D-glucuronic acid and N-acetyl-D-glucosamine.

“Hydrolytically degradable” refers to a crosslink that contains covalent bonds that dissociate due to nucleophilic attack of a water molecule. The dissociation may be catalyzed in the presence of an enzyme.

“Extent of chemical modification” refers to the percent of repeat units of a polymer that are modified with a new chemical group. For HA, the repeat unit is the disaccharide composed of D-glucuronic acid and N-acetyl-D-glucosamine, linked via alternating β-(1→4) and β-(1→3) glycosidic bonds.

“Extent of crosslinking” refers to the percent of repeat units on a polymer that are bound to another polymer or linker molecule through covalent or non-covalent interactions. For HA, the repeat unit is the disaccharide composed of D-glucuronic acid and N-acetyl-D-glucosamine, linked via alternating β-(1→4) and β-(1→3) glycosidic bonds.

“Fraction” is a subset of HA polymers that can be identified by differences in molecular weight, extent or type of chemical crosslinking, extent or type of chemical modification or crosslink density. These subsets may have a distribution around these properties but can be identified through a non-zero difference in the means of their distributions. These subsets may be produced separately through different chemical synthesis or formulation process steps.

“Molecular weight” is a measure of the size of a polymer. As used herein, the term “molecular weight” refers to the number average molecular weight of a polymer as determined by gel permeation chromatography in aqueous buffer. When used to describe an HA polymer, molecular weight refers to the linear uncrosslinked HA.

“Functionality” refers to the number of chemical groups per molecule that are capable of crosslinking to another molecule.

“Storage modulus” or (G′) is a measure of stored energy under force and represents the elastic portion of the complex modulus of viscoelastic materials. G′ can be determined by oscillatory rheology.

“Loss modulus” or (G″) is a measure of energy dissipation under force and represents the viscous portion of the complex modulus of viscoelastic materials. G″ can be determined by oscillatory rheology.

“Physiological buffer” is an aqueous buffer with a pH and ionic concentrations found in the body. Phosphate buffered saline (PBS) which comprises 137 mM NaCl, 2.7 mM KCl, 10 mM Na2HPO4, 1.8 mM KH2PO4, pH 7.4 is a commonly used physiological buffer.

“Solubilize” refers to the process of an insoluble material going into solution or dissolving into the surrounding liquid.

“Drug” is any chemical substance of known structure that when administered to a living organism, produces a biological effect.

“Medicament” is any chemical substance used for medical treatment.

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C” or “A, B, and C,” means any individual one of A, B or C as well as any combination thereof.

Embodiments comprise compositions comprising molecules linked to hyaluronic acid. Embodiments comprise compositions comprising molecules and drugs linked to hyaluronic acid. Embodiments comprise linear hyaluronic acid polymers and more than one linear hyaluronic acid polymers bound to one another. The linkage to hyaluronic acid may provide enhanced control of molecule administration in the body and extend the duration of treatment to tissues in the body.

Embodiments comprise one molecule linked to hyaluronic acid. Embodiments comprise one medicament or drug linked to hyaluronic acid. Embodiments comprise more than one molecule linked to hyaluronic acid. Embodiments comprise more than one medicament or drug linked to hyaluronic acid. Embodiments comprise 1, 2, 3, 4, 5, 10, 20, 50, 100, 200, 500, 1,000, 2,000, 5,000 or 10,000 molecules linked to hyaluronic acid, or in a range between any two of the foregoing. Embodiments comprise 1, 2, 3, 4, 5, 10, 20, 50, 100, 200, 500, 1,000, 2,000, 5,000 or 10,000 medicaments or drugs linked to hyaluronic acid, or in a range between any two of the foregoing.

Embodiments comprise compositions comprising medicaments or drugs linked to acrylate modified hyaluronic acid. Embodiments comprise compositions comprising medicaments or drugs linked through reaction to acrylate groups on acrylate modified hyaluronic acid. Embodiments may comprise acrylate modifications to carboxylic acid groups, hydroxyl groups, primary hydroxy groups, periodate formed aldehyde groups, or N-acetyl groups on hyaluronic acid. Embodiments may comprise more than one chemical modification to hyaluronic acid. Embodiments include chemical modifications to carboxylic acid groups, hydroxyl groups, primary hydroxy groups, periodate formed aldehyde groups, or N-acetyl groups in addition to acrylate modification to one or more group on hyaluronic acid. Embodiments comprise compositions comprising medicaments or drugs linked to acrylate modified hyaluronic acid with less than 5 percent extent of linking. Embodiments comprise compositions comprising medicaments or drugs linked to acrylate modified hyaluronic acid with less than 4 percent extent of linking. Embodiments comprise compositions comprising medicaments or drugs linked to acrylate modified hyaluronic acid with less than 3 percent extent of linking. Embodiments comprise compositions comprising medicaments or drugs linked to acrylate modified hyaluronic acid with less than 2 percent extent of linking. Embodiments comprise compositions comprising medicaments or drugs linked to acrylate modified hyaluronic acid with less than 1 percent extent of linking. The extents of linking may be 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 percent of repeat disaccharides, or in a range between any two of the foregoing.

A composition herein may comprise a medicament or drug linked to hyaluronic acid or hyaluronic acid hydrogel. The medicament or drug may be selected from small molecules, radioisotopes, neurotoxins, peptides, polypeptides, proteins, antibodies, vaccines, cytokines, growth factors, polysaccharides, synthetic polymers, particles, DNA plasmids, mRNA, microRNAs, microRNA inhibitors, siRNAs, cells, cellular exosomes, cellular receptors, ligands to cellular receptors and other cellular components.

Embodiments include steroids and non-steroidal anti-inflammatory drugs (NSAIDs). NSAIDs may be selected from ibuprofen, naproxen, diclofenac, celecoxib, oxaprozin, aspirin, indomethacin, meloxicam, tenoxicam, lornoxicam, piroxicam, ketorolac, sulindac, tolmetin, etodolac, diflunisal, fenoprofen, flurbiprofen, ketoprofen, meclofenamate, nabumetnone, aceclofenac, and parecoxib.

Embodiments include chemotherapeutics. Chemotherapeutics may be selected from clophosphamide, methotrexate, 5-fluorouracil, vinorelbine, doxorubicin, docetaxel, bleomycin, vinblastine, dacarbazine, Mustine, vincristine, procarbazine, prednisolone, vincristine, bleomycin, etoposide, cisplatin, epirubicin, capecitabine, methotrexate, vinorelbine, folinic acid, oxaliplatin, gemcitabine, and ifosfamide.

Embodiments include radiopharmaceuticals. Radiopharmaceuticals may be selected from Technetium-99m, Yttrium-90, radium 223 dichloride, lutetium-177, lutetium Lu 177-dotatate, lutetium-177 [Lu-177] vipivotide tetraxetan, ibritumomab tiuxetan, tositumomab, iodine-131 tositumomab, gallium-68, gallium [ga-68] dotatate, actinium-225, actinium-225 (Ac-225) dotatate,

Embodiments include bioactive peptides. Embodiments include collagen and collagen-like peptides known to stimulate collagen production in the skin.

Embodiments include medicaments and drugs that are growth factors, chemokines, cytokines, enzymes, cellular receptors, angiogenic factors, anti-angiogenic factors, neurotoxins, hormones, neuropeptides, etc. Growth factors include but are not limited to fibroblast growth factors 1-23, insulin-like growth factors, transforming growth factor beta 1-3, platelet derived growth factors, epidermal growth factors, hepatocyte growth factors, connective tissue growth factors, etc. Embodiments include transforming growth factor beta 1 and engineered transforming growth factor beta 1 analogues. Neurotoxins include those derived from bacteria including but not limited to botulinum toxins types A, B, C1, C2, D, E, F and G, letibotulinumtoxinA, onabotulinumtoxinA, abobotulinumtoxinA, daxibotulinumtoxinA, daxibotulinumtoxinA-lanm, and engineered analogues of botulinum toxins. Neurotoxins may include those derived from animal venom including but not limited to protoxin-II derived from tarantula venom and engineered analogues of protoxin-II. Neurotoxins may include synthetic neurotoxins. Anti-angiogenic factors may include anti-vascular endothelial growth factor (VEGF) agents including but not limited to antibodies and antibody fragments that bind VEGF such as bevacizumab, ranibizumab, aflibercept, etc.

Embodiments include drugs and medicaments that are greater than 10 Da, 100 Da, 1 kDa, 5 kDa, 10 kDa, 100 kDa, 500 kDa, 1 MDa, 2 MDa between any one of 10 Da, 100 Da, 1 kDa, 5 kDa, 10 kDa, 100 kDa, 500 kDa, 1 MDa, 2 MDa, or 5 MDa or any one of 10 Da, 100 Da, 1 kDa, 5 kDa, 10 kDa, 100 kDa, 500 kDa, 1 MDa, 2 MDa, or 5 MDa.

Embodiments include peptides, oligopeptides, polypeptides and proteins. Embodiments include amino acid sequences and analogues derived from naturally occurring polypeptides. Embodiments include engineered polypeptides. Embodiments include peptides greater than 2 amino acids. Embodiments include oligopeptides between about 2 and 10 amino acids. Embodiments include polypeptides greater than about 10 amino acids. Embodiments include proteins greater than about 50 amino acids.

Embodiments include peptides, polypeptides and proteins greater than 100 Da, 500 Da, 1 kDa, 5 kDa, 10 kDa, 50 kDa, 100 kDa, 500 kDa, or 1 MDa, in a range between any two of 100 Da, 500 Da, 1 kDa, 5 kDa, 10 kDa, 50 kDa, 100 kDa, 500 kDa, 1 MDa, or 5 MDa, or any one of 100 Da, 500 Da, 1 kDa, 5 kDa, 10 kDa, 50 kDa, 100 kDa, 500 kDa, 1 MDa, or 5 MDa. Embodiments include polypeptides less than 5 kDa. Embodiments include polypeptides less than 100 kDa. Embodiments include polypeptides between 1 kDa and 100 kDa.

Embodiments include medicaments and drugs containing thiol, free thiol, or sulfhydryl groups or medicaments and drugs that are chemically modified to contain thiol, free thiol, or sulfhydryl groups. Embodiments include molecules containing cysteine, homocysteine and N-acetylcysteine. Embodiments include peptides, polypeptides and proteins containing cysteine, homocysteine and N-acetylcysteine. Embodiments include molecules containing glutathione. Embodiments include peptides, polypeptides and proteins containing glutathione. Embodiments include molecules containing methionine and taurine.

Embodiments may comprise chemically modified medicaments and drugs. Medicaments and drugs may be chemically modified to react with acrylate modified hyaluronic acid. Medicaments and drugs may be chemically modified to react with thiols. Chemical modifications include those known in the art including but not limited to acrylate, methacrylate, vinyl sulfone, acrylamide, acrylonitriles, maleimide, thiol, primary amine, carboxylic acid, norbornene, alkyne and azide. Embodiments may comprise Michael acceptors and nucleophiles that participate in Michael addition reactions. Embodiments may comprise click chemistry. Embodiments may comprise polypeptides with non-conical, synthetic or novel amino acids. Embodiments include reduced peptides, polypeptides and proteins with free thiol, sulfhydryl groups available for crosslinking. Peptides, polypeptides and proteins may be reduced with methods known in the art including but not limited to incubation with reducing agents including but not limited to dithiothreitol, tris(2-carboxyethyl) phosphine hydrochloride (TCEP), beta-mercaptoethanol (BME) and glutathione, etc. Embodiments may comprise chemically modifying amino acids in peptides and proteins.

Embodiments comprise more than one type of molecule linked to hyaluronic acid. One, two, three, four, five, six, seven, eight, nine, ten or more different molecules may be linked to hyaluronic acid. Embodiments may comprise hyaluronic acid with different molecules linked with the same chemistry and methods or through different chemistries and methods.

An embodiment comprises cleavable chemical linkers known in the art including but not limited to hydrazone bonds, disulfide bonds, esters, glucuronide bonds, peptides, and hydrolytically degradable chemistries. Hydrolytically degradable chemistries may be selected from one or more of lactic acid, poly-L-lactic acid, caprolactone, polycaprolactone, glycolic acid, polyglycolic acid, hydroxyethyl methacrylate, esters, thioesters, and anhydrides. An embodiment may comprise enzyme cleavable chemical linkers.

An embodiment comprises non-cleavable chemical linkers known in the art including but not limited to thioethers, maleimidocaproyl and amide bonds.

An embodiment comprises chemical linkers that react with thiols. An embodiment may comprise chemical linkers that react with acrylates. Embodiments include chemical linkers that introduce new chemical groups on the hyaluronic acid or hyaluronic acid network for drug and medicament linking by first reacting with thiols or acrylates on the hyaluronic acid or hyaluronic acid network. Chemical groups include those known in the art including but not limited to acrylate, methacrylate, vinyl sulfone, acrylamide, acrylonitriles, maleimide, thiol, primary amine, carboxylic acid, norbornene, alkyne and azide. Embodiments comprise Michael acceptors and nucleophiles that participate in Michael addition reactions. Embodiments comprise click chemistry.

The hyaluronic acid in any embodiment herein may include one or more chemically modified hyaluronic acid. The chemical modification may comprise at least one of an aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane, or hydrolytically degradable moieties. The hydrolytically degradable moieties may be selected from one or more of lactic acid, poly-L-lactic acid, caprolactone, polycaprolactone, glycolic acid, polyglycolic acid, hydroxyethyl methacrylate, thioester and anhydrides. Embodiments comprise hyaluronic acid with multiple types of chemical modifications. Embodiments comprise acrylate modified hyaluronic acid with additional types of chemical modifications.

An extent of chemical modification for a hyaluronic acid herein may be greater than 0.01%, 0.05%, 0.1%, 0.2%, 0.35%, 0.5%, 1%, 2%, 3%, 4%, or 5%, 10%, 20%, or 50%, in a range between and including any two of 0.01%, 0.05%, 0.1%, 0.2%, 0.35%, 0.5%, 1%, 2%, 3%, 4%, 5%, 10%, 20%, 50% or 100%, or one of 0.01%, 0.05%, 0.1%, 0.2%, 0.35%, 0.5%, 1%, 2%, 3%, 4%, 5%, 10%, 20%, 50% or 100%.

Embodiments comprise compositions comprising networks of hyaluronic acid. The networks may provide enhanced control of hyaluronic acid hydration and swelling to increase the mechanical effects hyaluronic acid into the body and extend the duration of treatment of tissues in the body. The hyaluronic acid in the networks may be in a collapsed or precipitated form. The hyaluronic acid networks may be precipitates. The hyaluronic acid in the networks may be in a water swollen form. The hyaluronic acid in the networks may be in a semi-swollen form. The hyaluronic acid networks may be hydrogels.

An embodiment comprises a composition comprising a hyaluronic acid network with hydrolytically degradable crosslinks. The hyaluronic acid networks may have low extents of hydrolytically degradable crosslinking. The extents of hydrolytically degradable crosslinking may be less than 5 percent of repeat disaccharides. The extents of hydrolytically degradable crosslinking may be less than 4 percent of repeat disaccharides. The extents of hydrolytically degradable crosslinking may be less than 3 percent of repeat disaccharides. The extents hydrolytically degradable of crosslinking may be less than 2 percent of repeat disaccharides. The extents of hydrolytically degradable crosslinking may be 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 percent of repeat disaccharides, or in a range between and including any two of the foregoing. Embodiments include medicaments and drugs linked to hydrogels with low extents of hydrolytically degradable crosslinking.

An embodiment comprises a composition comprising a hyaluronic acid hydrogel with hydrolytically degradable crosslinks. The hyaluronic acid hydrogels may have low extents of hydrolytically degradable crosslinking. The extents of hydrolytically degradable crosslinking may be less than 5 percent of repeat disaccharides. The extents of hydrolytically degradable crosslinking may be less than 4 percent of repeat disaccharides. The extents of hydrolytically degradable crosslinking may be less than 3 percent of repeat disaccharides. The extents hydrolytically degradable of crosslinking may be less than 2 percent of repeat disaccharides. The extents of hydrolytically degradable crosslinking may be 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 percent of repeat disaccharides, or in a range between and including any two of the foregoing. Embodiments include medicaments and drugs linked to hydrogel with low extents of hydrolytically degradable crosslinking.

An embodiment comprises a composition comprising a hyaluronic acid hydrogel crosslinked with disulfide bonds. The extent of crosslinking may be less than 5 percent of repeat disaccharides. The extent of crosslinking may be less than 4 percent of repeat disaccharides. The extent of crosslinking may be less than 3 percent of repeat disaccharides. The extent of crosslinking may be less than 2 percent of repeat disaccharides. The extent of crosslinking may be 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 percent of repeat disaccharides, or in a range between and including any two of the foregoing. Embodiments include medicaments and drugs linked to hydrogel with low extents of disulfide crosslinking.

An embodiment comprises a composition comprising a hyaluronic acid hydrogel crosslinked with ester bonds. The extent of crosslinking may be less than 5 percent of repeat disaccharides. The extent of crosslinking may be less than 4 percent of repeat disaccharides. The extent of crosslinking may be less than 3 percent of repeat disaccharides. The extent of crosslinking may be less than 2 percent of repeat disaccharides. The extent of crosslinking may be 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 percent of repeat disaccharides, or in a range between and including any two of the foregoing. Embodiments include medicaments and drugs linked to hydrogel with low extents of ester crosslinking.

Embodiments comprise hyaluronic acid networks with more than one type of crosslinking or crosslink. An embodiment comprises a composition comprising a hyaluronic acid network crosslinked with a combination of disulfide bonds and ester bonds. An embodiment comprises a composition comprising a hyaluronic acid hydrogel crosslinked with a combination of disulfide bonds and ester bonds.

An embodiment may comprise cleavable crosslinking chemistries known in the art including but not limited to hydrazone bonds, disulfide bonds, glucuronide bonds, peptides, and hydrolytically degradable chemistries. Hydrolytically degradable chemistries may be selected from one or more of ester, lactic acid, poly-L-lactic acid, caprolactone, polycaprolactone, glycolic acid, polyglycolic acid, hydroxyethyl methacrylate, thioesters, and anhydrides. An embodiment may comprise enzyme cleavable crosslinkers.

An embodiment comprises a composition comprising an acrylate modified hyaluronic acid crosslinked to form a hydrogel. The acrylate modified hyaluronic acid may have low extents of chemical modification. Embodiments comprise acrylate modifications to carboxylic acid groups, hydroxyl groups, primary hydroxy groups, periodate formed aldehyde groups, or N-acetyl groups on hyaluronic acid. Embodiments comprise more than one chemical modification to hyaluronic acid. Embodiments include chemical modifications to carboxylic acid groups, hydroxyl groups, primary hydroxy groups, periodate formed aldehyde groups, or N-acetyl groups in addition to acrylate modification to one or more group on hyaluronic acid. The extents of acrylate modification may be less than 5 percent. The extents of acrylate modification may be less than 4 percent. The extents of acrylate modification may be less than 3 percent. The extents of acrylate modification may be less than 2 percent. The extents of acrylate modification may be 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 percent, or in a range between and including any two of the foregoing. Embodiments include medicaments and drugs linked to hydrogel made from crosslinking acrylate modified hyaluronic acid with low extents of chemical modification.

Embodiments comprise molecules linked to one or more hyaluronic acid repeat units on hyaluronic acid networks. The hyaluronic acid networks may comprise hydrogels. The molecules may be medicaments or drugs. The extent of linking may be greater than 0.005%, 0.01%, 0.03%, 0.05%, 0.1%, 0.3%, 0.5%, 1%, 3%, 5%, 10%, 20%, or 50%, any one of 0.005%, 0.01%, 0.03%, 0.05%, 0.1%, 0.3%, 0.5%, 1%, 3%, 5%, 10%, 20%, or 50%, or between and including any two of 0.005%, 0.01%, 0.03%, 0.05%, 0.1%, 0.3%, 0.5%, 1%, 3%, 5%, 10%, 20%, 50% or 100%. An extent of crosslinking for a hyaluronic acid network herein may be greater than 0.01%, 0.05%, 0.1%, 0.2%, 0.35%, 0.5%, 1%, 2%, 3%, 4%, or 5%, in a range between and including any two of 0.01%, 0.05%, 0.1%, 0.2%, 0.35%, 0.5%, 1%, 2%, 3%, 4%, or 5%, or one of 0.01%, 0.05%, 0.1%, 0.2%, 0.35%, 0.5%, 1%, 2%, 3%, 4%, or 5%. An extent of crosslinking for a hyaluronic acid network herein may be greater than 0.005%, 0.01%, 0.03%, 0.05%, 0.1%, 0.3%, 0.5%, 1%, 3%, 5%, 10%, 20%, or 50%, any one of 0.005%, 0.01%, 0.03%, 0.05%, 0.1%, 0.3%, 0.5%, 1%, 3%, 5%, 10%, 20%, or 50%, or between and including any two of 0.005%, 0.01%, 0.03%, 0.05%, 0.1%, 0.3%, 0.5%, 1%, 3%, 5%, 10%, 20%, 50% or 100%.

Embodiments comprise linked medicament and drug concentrations greater than 0.01 ng/ml, 0.1 ng/ml, 0.5 ng/ml, 1 ng/mL, 1 microgram/mL, 1 milligram/mL, 10 milligram/mL, 100 milligram/mL, between 0.01 ng/ml, 0.1 ng/ml, 0.5 ng/ml, 1 ng/ml, 1 microgram/mL, 1 milligram/mL, 10 milligram/mL, 100 milligram/mL, and 500 milligram/mL, or any one of 0.01 ng/ml, 0.1 ng/ml, 0.5 ng/ml, 1 ng/mL, 1 microgram/mL, 1 milligram/mL, 10 milligram/mL, 100 milligram/mL, and 500 milligram/mL.

Embodiments comprise multifunctional medicament or drug crosslinkers. Embodiments comprise medicament and drugs with multifunctional thiol groups used to crosslink acrylate modified hyaluronic acid with low extents of chemical modification. Embodiments comprise peptide medicament and drugs with multifunctional thiol groups used to crosslink acrylate modified hyaluronic acid. Embodiments comprise polypeptide medicament and drugs with multifunctional thiol groups used to crosslink acrylate modified hyaluronic acid. Embodiments comprise protein medicament and drugs with multifunctional thiol groups used to crosslink acrylate modified hyaluronic. Embodiments include peptide, oligopeptide, polypeptide and protein crosslinkers. Embodiments include amino acid sequences and analogues derived from naturally occurring polypeptides. Embodiments include engineered polypeptides. Embodiments include peptides greater than 2 amino acids. Embodiments include oligopeptides between about 2 and 10 amino acids. Embodiments include polypeptides greater than about 10 amino acids. Embodiments include proteins greater than about 50 amino acids.

An embodiment comprises a method of making a network of hyaluronic acid. The method may comprise chemically modifying hyaluronic acid with acrylate groups at less than 5 percent extent of chemical modification. The method may comprise chemically modifying hyaluronic acid with acrylate groups at less than 4 percent extent of chemical modification. The method may comprise chemically modifying hyaluronic acid with acrylate groups at less than 3 percent extent of chemical modification. The method may comprise chemically modifying hyaluronic acid with acrylate groups at less than 2 percent extent of chemical modification. The acrylate modified hyaluronic acid may then be mixed with multifunctional thiol containing molecules and incubated for extended times to facilitate crosslinking. The multifunctional thiol crosslinker may comprise a medicament or drug. Complimentary thiol modified hyaluronic acids may be used to facilitate crosslinking. The method may comprise mixing to facilitate crosslinking reactions. The method may comprise precipitations to facilitate crosslinking reactions. The method may further comprise adding chemical initiators to the acrylate modified hyaluronic acid. The chemical initiators may initiate crosslinking. The method may comprise adding at least one of light or heat to initiate crosslinking. The initiation of crosslinking may be conducted following mixing crosslinkers with acrylate modified hyaluronic acid. The final product may comprise a microparticle powder. The method may further comprise reconstituting the products in aqueous buffer.

An embodiment comprises a method of making a network of hyaluronic acid and linked molecules. The method may comprise first linking molecules to hyaluronic acid. Molecules may be linked to hyaluronic acid through methods known in the art including but not limited to reaction with modifications to carboxylic acid groups, hydroxyl groups, primary hydroxy groups, periodate formed aldehyde groups, or N-acetyl groups. The method may then comprise chemically modifying hyaluronic acid with acrylate groups at less than 5 percent extent of chemical modification. The method may comprise chemically modifying hyaluronic acid with acrylate groups at less than 4 percent extent of chemical modification. The method may comprise chemically modifying hyaluronic acid with acrylate groups at less than 3 percent extent of chemical modification. The method may comprise chemically modifying hyaluronic acid with acrylate groups at less than 2 percent extent of chemical modification. The method may then comprise linking molecules to the acrylate modified hyaluronic acid. The molecules may be linked through reaction with the acrylate groups or to chemical linkers reacted to acrylate groups. The acrylate modified hyaluronic acid may then be mixed with multifunctional crosslinking molecules and incubated for extended times to facilitate crosslinking. The multifunctional crosslinker may comprise a medicament or drug. Complimentary modified hyaluronic acids may be used to facilitate crosslinking. The method may comprise mixing to facilitate crosslinking reactions. The method may comprise precipitations to facilitate crosslinking reactions. The method may further comprise adding chemical initiators to the acrylate modified hyaluronic acid. The chemical initiators may initiate crosslinking. The method may comprise adding at least one of light or heat to initiate crosslinking. The initiation of crosslinking may be conducted following mixing crosslinkers with acrylate modified hyaluronic acid. Molecules may then be linked to the network of hyaluronic acid. The method may comprise linking molecules to the hyaluronic acid network after crosslinking. The method may comprise linking molecules to excess acrylate groups on the hyaluronic acid after crosslinking. The method may comprise linking molecules to excess crosslinking groups on the crosslinker after crosslinking. The method may comprise linking molecules to excess thiol groups on the crosslinker after crosslinking. The method may comprise a first step of reacting a chemical linker to excess acrylate or thiol groups on the crosslinked hyaluronic acid network, and then reacting a molecule to the chemical linker. The molecule may be a medicament or a drug. The molecule may be chemically modified for reaction to the acrylate modified hyaluronic acid, crosslinker, thiol containing crosslinker or chemical linker reacted to hyaluronic acid or crosslinker. The method may comprise swelling a solution of molecules into a powder comprising hyaluronic acid networks for linking. The molecules may be in aqueous or non-aqueous solvents prior to swelling into hyaluronic acid networks for linking. The method may comprise incubations for extended periods of time to facilitate linking reactions. The method may comprise adding heat or light to facilitate linking reactions. The method may comprise altering pH to facilitate linking reactions. The method may comprise mixing to facilitate linking reactions. The method may comprise precipitations to facilitate linking reactions.

An embodiment comprises a composition made by dissolving sodium hyaluronate in water at a concentration of 5 mg/mL. The aqueous solution of sodium hyaluronate is maintained at a pH of 7.0 to 9.0 using 0.5 M NaOH after the addition of acrylic anhydride. The reaction is allowed to proceed for 18 hrs. Embodiments include sodium hyaluronate concentrations greater than 100 micrograms/mL, 1 mg/mL, 2 mg/mL, 5 mg/mL, 10 mg/mL, or 20 mg/mL, between and including any two of 100 micrograms/mL, 1 mg/mL, 2 mg/mL, 5 mg/mL, 10 mg/mL, 20 mg/mL, 40 mg/mL or 100 mg/mL, or any of 100 micrograms/mL, 1 mg/mL, 2 mg/mL, 5 mg/mL, 10 mg/mL, 20 mg/mL, 40 mg/mL, or 100 mg/mL. Solution pH values include greater than pH 6, 6.5, 7, 7.5, 8, 8.5, 9, 9.5, 10, 10.5, between and including any two of pH 6, 6.5, 7, 7.5, 8, 8.5, 9, 9.5, 10, 10.5, or 11, or any of pH 6, 6.5, 7, 7.5, 8, 8.5, 9, 9.5, 10, 10.5, or 11. Molar ratios of acrylic anhydride to the repeat unit of sodium hyaluronate include those greater than 0.002, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.25, 0.5, 0.75 and 1. Molar ratios of acrylic anhydride to the repeat unit of sodium hyaluronate include those between and including any two of 0.002, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.25, 0.5, 0.75, 1.0, 1.5, 2 or 10. Molar ratios of acrylic anhydride to the repeat unit of sodium hyaluronate include any of 0.002, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.25, 0.5, 0.75, 1.0, 1.5, 2 or 10. Embodiments include purifying and drying the acrylate modified HA.

The hyaluronic acid molecular weights in embodiments herein may be greater than 10 kDa, 20 kDa, 50 kDa, 100 kDa, 200 kDa, 500 kDa, 700 kDa, 1000 kDa, 2000 kDa, 3000 kDa, or 4000 kDa, in a range between and including any two of 10 kDa, 20 kDa, 50 kDa, 80 kDa, 100 kDa, 200 kDa, 500 kDa, 700 kDa, 1000 kDa, 2000 kDa, 3000 kDa, or 4000 kDa, or one of 80 kDa, 200 kDa, 500 kDa, 700 kDa, 1000 kDa, 2000 kDa, 3000 kDa, or 4000 kDa.

Embodiments include compositions made by dissolving the acrylate modified hyaluronic acid in aqueous buffer and adding thiol containing crosslinkers. Concentrations of acrylate modified hyaluronic acid include those greater than 1, 2, 3, 4, 5, 6, 7, 8, 9, 10, 15, 20, 25, 30, 35, 40, 50, 60, 70 and 80 mg/mL. Concentrations of acrylate modified hyaluronic acid include those between and including any two of 1, 2, 3, 4, 5, 6, 7, 8, 9, 10, 15, 20, 25, 30, 35, 40, 50, 60, 70, 80, 100 and 200 mg/mL. Concentrations of acrylate modified hyaluronic acid include any one of 1, 2, 3, 4, 5, 6, 7, 8, 9, 10, 15, 20, 25, 30, 35, 40, 50, 60, 70 and 80 mg/mL. Aqueous buffer pH values include those greater than 5, 5.5, 6, 6.5, 7, 7.5, 8, 8.5, 9, 9.5, 10, and 10.5. Aqueous buffer pH values include those between and including any two of 5, 5.5, 6, 6.5, 7, 7.5, 8, 8.5, 9, 9.5, 10, 10.5, and 11. Aqueous buffer pH values include any one of 5, 5.5, 6, 6.5, 7, 7.5, 8, 8.5, 9, 9.5, 10, 10.5, and 11. Incubation temperatures include those greater than 4, 10, 15, 20, 25, 30, 35, 40, 45, 50 and 60 Celsius. Incubation temperatures include those between and including any two of 4, 10, 15, 20, 25, 30, 35, 40, 45, 50 and 60 Celsius. Incubation temperatures include any one of 4, 10, 15, 20, 25, 30, 35, 40, 45, 50 and 60 Celsius. Incubation times include those greater than 1, 2, 3, 4, 5, 6, 7, 8, 9, 10, 11, 12, 13 and 14 days. Incubation times include any one of 1, 2, 3, 4, 5, 6, 7, 8, 9, 10, 11, 12, 13 and 14 days. Embodiments include Michael-addition catalysts including those known in art, including but not limited to triethanolamine and tris(2-carboxyethyl) phosphine. Embodiments include mixing protocols to facilitate the crosslinking reactions. Embodiments include precipitations to facilitate the crosslinking reactions.

Embodiments include thiol containing crosslinkers with a functionality greater than 1, 2, 3, 4, 5, 10, 20, 25, 50, 100, 200, or 500. Embodiments include thiol containing crosslinkers with a functionality between and including any two of 1, 2, 3, 4, 5, 10, 20, 25, 50, 100, 200, or 500. Embodiments include thiol containing crosslinkers with a functionality of any one of 2, 3, 4, 5, 10, 20, 25, 50, 100, 200, or 500. Embodiments include multifunctional thiol crosslinkers that are medicaments or drugs.

Embodiments include thiol containing crosslinkers comprised of polymers including those known in the art, including but not limited to hyaluronic acid, polyethylene glycol, carboxymethylcellulose, proteins, and peptides.

Embodiments include free-radical initiated crosslinking of acrylate groups using initiators and methods known in the art including but not limited to redox initiators, light initiated polymerizations, light initiated click-reactions, etc.

The composition may comprise multiple fractions of hyaluronic acid. Each fraction may be made by separate iterations of this method. Different fractions may comprise networks of hyaluronic acid may be one or more iteration of this method combined with networks of hyaluronic acid made by another method(s). The other method(s) may be one or more method herein, or any hyaluronic acid crosslinking method.

The polymer networks may comprise microparticles having between 10 microns and 2 millimeter in diameter when reconstituted in aqueous buffer. The microparticle diameter may be selected from those described below. Microparticle diameters in embodiments herein may include 10 microns, 35 microns, 50 microns, 75 microns, 100 microns, 200 microns, 300 microns, 400 microns, 500 microns, 600 microns, 700 microns, 800 microns, 900 microns, 1 mm, 2 mm after reconstitution in physiologic buffer. Microparticle sizes in embodiments herein may include a range of sizes between and including any two of 10 microns, 35 microns, 50 microns, 75 microns, 100 microns, 200 microns, 300 microns, 400 microns, 500 microns, 600 microns, 700 microns, 800 microns, 900 microns, 1 mm, 2 mm after reconstitution in physiologic buffer. Microparticle sizes in embodiments herein may include a range of sizes between and including 50 microns and 250 microns. Microparticle sizes in embodiments herein may include a range of sizes between and including 250 microns and 2 mm. The microparticles may be hydrogels.

An embodiment comprises a particle composition wherein hydrogels are fragmented to form particles. Embodiments include methods that set particle size prior to hydrogel crosslinking including but not limited to emulsions, coacervates and microfluidic techniques. An embodiment includes milling or sizing the dried hydrogel to control particle size.

Embodiments include formulations comprising more than one hyaluronic acid network or fraction that solubilize at different rates. Embodiments include formulations with 2, 3, 4, 5, 6, 7, 8, 9 or 10 separate networks or fractions.

An embodiment comprises a composition comprising any one or more network herein. The compositions may comprise two or more separate hyaluronic acid networks. The two or more networks may hydrate and/or swell at different rates. A hyaluronic acid network herein may swell and uptake 1, 2, 5, 10, 20, 50, 100, or 200 times its dry mass with water 24 hrs after being reconstituted with excess aqueous buffer. A hyaluronic acid network herein may swell and uptake between and including any two of 1, 2, 5, 10, 20, 50, 100, or 200 times its dry mass with water 24 hrs after being reconstituted with excess aqueous buffer. The hyaluronic acid network may be in a water swollen or hydrogel form. The hyaluronic acid network may be in a collapsed or precipitated form. Water uptake may be measured by centrifuging the material suspended in aqueous buffer, removing the supernatant and weighing the hydrated pellet.

The composition may be optically transparent upon reconstitution in aqueous buffer. The composition may have optical opacity due to refraction of light on the microparticles surfaces.

An embodiment comprises a network of hyaluronic acid in the form of a dry powder. An embodiment comprises a network of hyaluronic acid in the form of a dry microparticle powder. An embodiment comprises a network of hyaluronic acid in the form of a dry nanoparticle powder. An embodiment comprises a network of hyaluronic acid in the form of a swollen gel. An embodiment comprises a network of hyaluronic acid in the form of a partially swollen gel. The gel may be swollen with water or aqueous buffer. The gel may be partially swollen with non-aqueous solvents. The gel may be partially swollen with a combination of aqueous and non-aqueous solvents. An embodiment comprises a network of hyaluronic acid in the form of a hydrogel. An embodiment comprises a network of hyaluronic acid in the form of microparticles. An embodiment comprises a network of hyaluronic acid in the form of nanoparticles. Nanoparticles may have an average diameter of greater than 1 nm, 10 nm, or 100 nm, or between and including any two of 1 nm, 10 nm, 100 nm or 1 micron, or any of 1 nm, 10 nm, 100 nm or 1 micron.

Hyaluronic acid networks in embodiments herein may solubilize in phosphate buffered saline at 37° C., pH 7.4 over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, 180, or 365 days or any one of 2, 3, 4, 5, 6, 7, 8, 9, 10, 20, 30, 40, 50, 60, 70, 80, 90, 180 or 365 days. In one aspect, one fraction solubilizes in 2 to 7 days; a second fraction solubilizes in 10 to 14 days; and a third fraction solubilizes in 17 to 21 days to mimic a three-injection regime. In an embodiment, one fraction solubilizes in 2 to 7 days; a second fraction solubilizes in 10 to 14 days; a third fraction solubilizes in 17 to 21 days; a fourth fraction solubilizes in 24 to 28 days and a fifth fraction solubilizes in 31 to 35 days to mimic a five-injection regime. In an embodiment, one fraction solubilizes with two weeks; a second fraction solubilizes within four weeks; and a third fraction solubilizes with 6 weeks. In another aspect, one fraction solubilizes within one month; a second fraction solubilizes within two months; and a third fraction solubilizes over three months. In an embodiment, one fraction solubilizes within one month; a second fraction solubilizes within two months; and a third fraction solubilizes over three months.

Hyaluronic acid networks in embodiments herein may maintain exogenous HA at concentrations greater than 100 μg/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days, or any one of 2, 3, 4, 5, 6, 7, 8, 9, 10, 20, 30, 40, 50, 60, 70, 80, 90, or 180 days.

Hyaluronic acid networks in embodiments herein may maintain exogenous hyaluronic acid at concentrations greater than 1 mg/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days, or any one of 2, 3, 4, 5, 6, 7, 8, 9, 10, 20, 30, 40, 50, 60, 70, 80, 90, or 180 days.

Hyaluronic acid networks in embodiments herein may maintain exogenous hyaluronic acid at concentrations greater than 100 μg/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days, or any one of 2, 3, 4, 5, 6, 7, 8, 9, 10, 20, 30, 40, 50, 60, 70, 80, 90, or 180 days.

Hyaluronic acid networks in embodiments herein may maintain exogenous hyaluronic acid at concentrations greater than 10 mg/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days, or any one of 2, 3, 4, 5, 6, 7, 8, 9, 10, 20, 30, 40, 50, 60, 70, 80, 90, or 180 days.

Hyaluronic acid networks in embodiments herein may increase the storage modulus of tissue and fluids in the body by a value greater than 1 Pa, 10 Pa, 20 Pa, 30 Pa, 40 Pa, 50 Pa, 100 Pa, or 500 Pa, in a range between and including any two of 1 Pa, 10 Pa, 20 Pa, 30 Pa, 40 Pa, 50 Pa, 100 Pa, 500 Pa, or 1 kPa, or any one of 1 Pa, 10 Pa, 20 Pa, 30 Pa, 40 Pa, 50 Pa, 100 Pa, 500 Pa, or 1 kPa.

Hyaluronic acid networks in embodiments herein may increase the loss modulus of tissue and fluids in the body by a value greater than 1 Pa, 10 Pa, 20 Pa, 30 Pa, 40 Pa, 50 Pa, 100 Pa, or 500 Pa, in a range between and including any two of 1 Pa, 10 Pa, 20 Pa, 30 Pa, 40 Pa, 50 Pa, 100 Pa, 500 Pa, or 1 kPa, or any one of 1 Pa, 10 Pa, 20 Pa, 30 Pa, 40 Pa, 50 Pa, 100 Pa, 500 Pa, or 1 kPa.

Hyaluronic acid networks in embodiments herein may increase the viscosity of tissue and fluids in the body by a value greater than 1 Pa, 10 Pa, 20 Pa, 30 Pa, 40 Pa, 50 Pa, 100 Pa, or 500 Pa, in a range between and including any two of 1 Pa, 10 Pa, 20 Pa, 30 Pa, 40 Pa, 50 Pa, 100 Pa, 500 Pa, or 1 kPa, or any one of 1 Pa, 10 Pa, 20 Pa, 30 Pa, 40 Pa, 50 Pa, 100 Pa, 500 Pa, or 1 kPa.

A composition herein may comprise a pharmaceutically acceptable carrier. Embodiments include carriers of uncrosslinked or crosslinked hyaluronic acid in aqueous buffer. In an embodiment, the hyaluronic acid carrier concentration is greater than 1 mg/mL. Embodiments include hyaluronic acid carrier concentrations between 10, 20, 40 and 80 mg/mL.

Embodiments may include formulations comprising one or more HA network herein.

A method may comprise injecting the reconstituted products of a method of making herein onto or into a body of a subject. The subject may be a human. The subject may be a mammal. The subject may be horse. The subject may be a dog. The injecting into a body may comprise applying to the skin or eye of a subject for hydration or anti-inflammation. The injecting into a body may comprise injecting into the synovial fluid or fat pad to treat osteoarthritis. The injecting into a body may comprise injecting into the synovial fluid or fat pad to treat synovitis. The injecting into a body may comprise injecting into the pericardial fluid to treat heart disease. The injecting into a body may comprise injecting into the spine to treat degenerative disc disease. The injecting into a body may comprise injecting into the vitreous of the eye to treat ocular disease. The injecting into a body may comprise injecting into solid tumors or resected tumor sites to treat cancer. The injecting into a body may comprise injecting into the dermis or subcutaneous fat to provide tissue volume or reduce the appearance of wrinkles. The injecting into a body may comprise injecting into the scalp to treat baldness. The injecting into a body may comprise injecting into an artery or vein for systemic delivery.

A method may comprise administering reconstituted composition comprising an acrylate modified hyaluronic acid with linked molecules herein onto or into a body of a subject. The subject may be a human. The subject may be a mammal. The subject may be horse. The subject may be a dog. The administration onto a body may comprise applying to the subject for hydration or anti-inflammation. The administration onto a body may comprise applying to the skin, vagina, or eye of a subject for hydration or anti-inflammation. The administration into a body may comprise injecting into the synovial fluid or fat pad to treat osteoarthritis. The administration into a body may comprise injecting into the synovial fluid or fat pad to treat synovitis. The administration into a body may comprise injecting into the pericardial fluid to treat heart disease. The administration into a body may comprise injecting into the spine to treat degenerative disc disease. The administration into a body may comprise injecting into the vitreous of the eye to treat ocular disease. The administration into a body may comprise injecting into solid tumors or resected tumor sites to treat cancer. The administration into a body may comprise injecting into the dermis or subcutaneous fat to provide tissue volume or reduce the appearance of wrinkles. The administration into a body may comprise injecting into the scalp to treat baldness. The administration into a body may comprise injecting into an artery or vein for systemic delivery.

A method may comprise administering reconstituted composition comprising a hyaluronic acid hydrogel with ester crosslinks herein onto or into a body of a subject. The subject may be a human. The subject may be a mammal. The subject may be a horse. The subject may be a dog. The hyaluronic acid hydrogel may be microparticles. The hyaluronic acid hydrogel may be nanoparticles. The administration onto a body may comprise applying to the subject for hydration or anti-inflammation. The administration onto a body may comprise applying to the skin, vagina, or eye of a subject for hydration or anti-inflammation. The administration into a body may comprise injecting into the synovial fluid or fat pad to treat joint inflammation or osteoarthritis. The administration into a body may comprise injecting into the synovial fluid or fat pad to treat synovitis. The administration into a body may comprise injecting into the pericardial fluid to treat heart disease. The administration into a body may comprise injecting into the spine to treat degenerative disc disease. The administration into a body may comprise injecting into the vitreous of the eye to treat ocular disease. The administration into a body may comprise injecting into solid tumors or resected tumor sites to treat cancer. The administration into a body may comprise injecting into the dermis or subcutaneous fat to provide tissue volume or reduce the appearance of wrinkles. The administration into a body may comprise injecting into the scalp to treat baldness. The administration into a body may comprise injecting into an artery or vein for systemic delivery.

A method may comprise injecting 2 mL of reconstituted hydrogel microparticles into the synovial cavity of a subject via intra-articular injection. Methods may comprise injecting more than 2 mL of reconstituted hydrogel microparticles into the synovial cavity via intra-articular injection. Methods may comprise injecting 3 mL of reconstituted hydrogel microparticles into the synovial cavity via intra-articular injection. Methods may comprise injecting 4 mL of reconstituted hydrogel microparticles into the synovial cavity via intra-articular injection. Methods may comprise injecting 5 mL of reconstituted hydrogel microparticles into the synovial cavity via intra-articular injection. Methods may comprise injecting 6 mL of reconstituted hydrogel microparticles into the synovial cavity via intra-articular injection. Methods may comprise injecting between 2 mL and 6 mL into the synovial cavity. The subject may be a human. The subject may be horse. The subject may be a dog. The injecting into a body may comprise injecting into the synovial fluid, synovial cavity, or fat pad to treat synovitis or osteoarthritis.

Methods may comprise reconstituting microparticles at 10 mg/ml prior to injecting into the synovial cavity of a subject. Methods may comprise reconstituting microparticles at 20 mg/mL prior to injecting into the synovial cavity. Methods may comprise reconstituting microparticles at greater than 20 mg/mL prior to injecting into the synovial cavity. Methods may comprise reconstituting microparticles at greater than 20 mg/ml, 30 mg/mL, 40 mg/mL, 50 mg/mL, 60 mg/mL, 70 mg/mL, or 80 mg/mL, between and including 20 mg/mL, 30 mg/mL, 40 mg/mL, 50 mg/mL, 60 mg/mL, 70 mg/mL, and 80 mg/mL, or any one of 20 mg/mL, 30 mg/mL, 40 mg/mL, 50 mg/mL, 60 mg/mL, 70 mg/mL, or 80 mg/mL prior to injecting into the synovial cavity. The subject may be a human. The subject may be horse. The subject may be a dog. The injecting into a body may comprise injecting into the synovial fluid, synovial cavity or fat pad to treat synovitis or osteoarthritis.

Reconstituted microparticles in embodiments herein may have diameters between 10 microns and 2 millimeter. Microparticle sizes in embodiments herein may include a range of sizes between 50 microns and 250 microns. Microparticle sizes in embodiments herein may include a range of sizes between 250 microns and 2 mm.

The composition may be soft upon reconstitution in aqueous buffer. The composition may be soft to such a degree that it has a storage modulus of less than 100 kPa. The composition may be soft to such a degree that it has a storage modulus of less than 50 kPa. The composition may be soft to such a degree that it has a storage modulus of less than 10 kPa. The composition may be soft to such a degree that it has a storage modulus of less than 5 kPa. The composition may be soft to such a degree that it has a storage modulus of less than 2 kPa. The composition may be soft to such a degree that it has a storage modulus of less than 1 kPa. The composition may be soft to such a degree that it has a storage modulus of between 10 Pa and 10 kPa. The composition may be soft to such a degree that it has a storage modulus of between and including 100 Pa and 1 kPa. The composition may be soft to such a degree that it has a storage modulus of between and including 300 Pa and 2 kPa. The composition may be used in applications where a softer material is desired. For example in synovial fluid, pericardial fluid, vitreous of the eye, skin, vaginal fluid, or nucleus pulposus. The composition may have tan delta values less than 2 as measured with oscillatory rheology. The composition may have tan delta values less than 1 as measured with oscillatory rheology.

An embodiment comprises a composition comprising any composition or combination of compositions described herein.

An embodiment comprises a method of treatment. The method comprises administering a composition herein to a subject in need thereof. The administering may be by injection to a site if interest. The injection may be via syringe. The injection may be via a catheter. The site of interest may be synovial fluid, pericardial fluid, topical wound bed, diabetic ulcer bed, tumor microenvironment, nucleus pulposus, vitreous humor, surgical intervention site, vaginal canal, rectum, skin dermis, subcutaneous fat, scalp, cerebrospinal fluid or bloodstream. The subject may be human. The subject may be a dog, horse or cat.

The subject may be a mammal. The method may comprise administering a composition herein comprising a hyaluronic acid network(s) to the body via injection. The injected hyaluronic acid network(s) may provide mechanical support from swelling hyaluronic acid as the crosslinks hydrolyze. The injected hyaluronic acid network(s) may provide sustained molecule release as the crosslinks hydrolyze.

An embodiment comprises a formulation comprising at least one of any network, hydrogel or composition disclosed herein.

Embodiments include any method of making a network, hydrogel or composition disclosed herein.

An embodiment comprises any method of treatment disclosed herein.

Networks of HA in embodiments herein may have a concentration of HA after reconstitution in physiological buffer greater than 2 mg/mL, 5 mg/mL, 10 mg/mL, 20 mg/ml, 30 mg/mL, 40 mg/mL, 50 mg/mL, 60 mg/mL, 70 mg/mL, 80 mg/mL, 90 mg/mL or 100 mg/mL, in a range between and including any two of 2 mg/mL, 5 mg/mL, 10 mg/mL, 20 mg/ml, 30 mg/mL, 40 mg/mL, 50 mg/mL, 60 mg/mL, 70 mg/mL, 80 mg/mL, 90 mg/mL, 100 mg/mL, or 200 mg/mL, or one of 2 mg/mL, 5 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, or 200 mg/mL.

A composition herein may further comprise at least one hyaluronidase inhibitor. The hyaluronidase inhibitor may prevent hyaluronidase mediated degradation of one or more of the HA fractions. The at least one hyaluronidase inhibitor may be separate from or embedded in one or more of the fractions. The hyaluronidase inhibitor(s) may be selected from sulfated polysaccharides. The sulfated polysaccharides may be but are not limited to heparin, heparan sulfate, chondroitin sulfate, and synthetically derived molecules. The synthetically derived molecules may be but are not limited to sulfated hyaluronic acid, dextran sulfate, and pentosan polysulfate. The sulfated polysaccharides may be partially or fully sulfated. The partially or fully sulfated polysaccharide described herein can be the pharmaceutically acceptable salt or ester thereof.

Any one or more composition herein may be used in a method of hydrating or lubricating. Any one or more composition herein may be used in a method of treating inflammation. Any one or more composition herein may be used in a method of treating vaginal dryness or atrophy. Any one or more composition herein may be used in a method of treating osteoarthritis. Any one or more composition herein may be used in a method of treating synovitis. Any one or more composition herein may be used in a method of treating ocular disease. Any one or more composition herein may be used in a method of treating cardiovascular disease. Any one or more composition herein may be used in a method of treating cancer. Any one or more composition herein may be used in a method of treating disc degeneration. Any one or more composition herein may be used in a method of treating diabetic ulcers. Any one or more composition herein may be used in a method of treating pain. Any one or more composition herein may be used in a method of reducing inflammation and pain following surgery. Any one or more composition herein may be used in a method to improve tissue healing following surgery or a traumatic injury. Any one or more composition herein may be used in a method to reduce the appearance of wrinkles. Any one or more composition herein may be used in a method to provide volume to the skin. Any one or more composition herein may be used in a method to stimulate tissue regeneration. Any one or more composition herein may be used in a method to stimulate hair growth. A method of treating may comprise administering one or more composition herein to one of the foregoing sites. The administering may be by injection. The injection may be via syringe, or via catheter.

An embodiment comprises the use of any composition herein for hydrating or lubricating. An embodiment comprises the use of any composition herein for treating inflammation. An embodiment comprises the use of any composition herein for treating vaginal dryness or atrophy. An embodiment comprises the use of any composition herein for treating osteoarthritis. An embodiment comprises the use of any composition herein for treating synovitis. An embodiment comprises the use of any composition herein for treating ocular disease. An embodiment comprises the use of any composition herein for treating cardiovascular disease. Any one or more composition herein may be used in a method of treating cancer. An embodiment comprises the use of any composition herein for treating disc degeneration. An embodiment comprises the use of any composition herein for treating diabetic ulcers. An embodiment comprises the use of any composition herein for treating pain. An embodiment comprises the use of any composition herein for reducing inflammation and pain following surgery. An embodiment comprises the use of any composition herein for improving tissue healing following surgery or a traumatic injury. Any one or more composition herein may be used in a method to reduce the appearance of wrinkles. Any one or more composition herein may be used in a method to provide volume to the skin. Any one or more composition herein may be used in a method to stimulate tissue regeneration. Any one or more composition herein may be used in a method to stimulate hair growth.

An embodiment comprises the use of two or more compositions herein in combination for hydrating or lubricating. An embodiment comprises the use of two or more compositions herein in combination for treating inflammation. An embodiment comprises the use of two or more compositions herein in combination for treating vaginal dryness or atrophy. An embodiment comprises the use of two or more compositions herein in combination for treating osteoarthritis. An embodiment comprises the use of two or more compositions herein in combination for treating synovitis. An embodiment comprises the use of two or more compositions herein in combination for treating ocular disease. An embodiment comprises the use of two or more compositions herein in combination for treating cardiovascular disease. An embodiment comprises the use of two or more compositions herein in combination to treat cancer. An embodiment comprises the use of two or more compositions herein in combination for treating disc degeneration. An embodiment comprises the use of two or more compositions herein in combination for treating diabetic ulcers. An embodiment comprises the use of two or more compositions herein in combination for treating pain. An embodiment comprises the use of two or more compositions herein in combination for reducing inflammation and pain following surgery. An embodiment comprises the use of two or more compositions herein in combination for improving tissue healing following surgery or a traumatic injury. An embodiment comprises the use of two or more compositions herein in combination to reduce the appearance of wrinkles. An embodiment comprises the use of two or more compositions herein in combination to provide a volume in the skin. An embodiment comprises the use of two or more compositions herein in combination to stimulate tissue regeneration. An embodiment comprises the use of two or more compositions herein in combination to stimulate hair growth.

Any one or more formulations herein may be used in a method of hydrating or lubricating. Any one or more formulation herein may be used in a method of treating inflammation. Any one or more formulation herein may be used in a method of treating vaginal dryness or atrophy. Any one or more formulation herein may be used in a method of treating osteoarthritis. Any one or more formulation herein may be used in a method of treating synovitis. Any one or more formulations herein may be used in a method of treating ocular disease. Any one or more formulations herein may be used in a method of treating cardiovascular disease. Any one or more formulations herein may be used in a method of treating cancer. Any one or more formulations herein may be used in a method of treating disc degeneration. Any one or more formulations herein may be used in a method of treating diabetic ulcers. Any one or more formulations herein may be used in a method of treating pain. Any one or more formulations herein may be used in a method of reducing inflammation and pain following surgery. Any one or more formulations herein may be used in a method to improve tissue healing following surgery or a traumatic injury. Any one or more formulations herein may be used in a method to reduce the appearance of wrinkles. Any one or more formulations herein may be used in a method to provide a volume in the skin. Any one or more formulations herein may be used in a method to stimulate tissue regeneration. Any one or more formulations herein may be used in a method to stimulate hair growth. A method of treating may comprise administering one or more formulations herein to one of the foregoing sites. The administering may be by injection. The injection may be via syringe, or via catheter.

An embodiment comprises the use of any formulation herein for hydrating or lubricating. An embodiment comprises the use of any formulation herein for treating inflammation. An embodiment comprises the use of any formulation herein for treating vaginal dryness or atrophy. An embodiment comprises the use of any formulation herein for treating osteoarthritis. An embodiment comprises the use of any formulation herein for treating synovitis. An embodiment comprises the use of any formulation herein for treating ocular disease. An embodiment comprises the use of any formulation herein for treating cardiovascular disease. An embodiment comprises the use of any formulation herein for treating cancer. An embodiment comprises the use of any formulation herein for treating disc degeneration. An embodiment comprises the use of any formulation herein for treating diabetic ulcers. An embodiment comprises the use of any formulation herein for treating pain. An embodiment comprises the use of any formulation herein for reducing inflammation and pain following surgery. An embodiment comprises the use of any formulation herein for improving tissue healing following surgery or a traumatic injury. An embodiment comprises the use of any formulation herein for reducing the appearance of wrinkles. An embodiment comprises the use of any formulation herein for producing a volume in the skin. An embodiment comprises the use of any formulation herein for stimulating tissue regeneration. An embodiment comprises the use of any formulation herein for stimulating hair growth.

An embodiment comprises the use of two or more formulations herein in combination for hydrating or lubricating. An embodiment comprises the use of two or more formulations herein in combination for treating inflammation. An embodiment comprises the use of two or more formulations herein in combination for treating vaginal dryness or atrophy. An embodiment comprises the use of two or more formulations herein in combination for treating osteoarthritis. An embodiment comprises the use of two or more formulations herein in combination for treating synovitis. An embodiment comprises the use of two or more formulations herein in combination for treating ocular disease. An embodiment comprises the use of two or more formulations herein in combination for treating cardiovascular disease. An embodiment comprises the use of two or more formulations herein in combination for treating cancer. An embodiment comprises the use of two or more formulations herein in combination for treating disc degeneration. An embodiment comprises the use of two or more formulations herein in combination for treating diabetic ulcers. An embodiment comprises the use of two or more formulations herein in combination for treating pain. An embodiment comprises the use of two or more formulations herein in combination for reducing inflammation and pain following surgery. An embodiment comprises the use of two or more formulations herein in combination for improving tissue healing following surgery or a traumatic injury. An embodiment comprises the use of two or more formulations herein in combination for reducing the appearance of wrinkles. An embodiment comprises the use of two or more formulations herein in combination for providing volume in the skin. An embodiment comprises the use of two or more formulations herein in combination for stimulating tissue regeneration. An embodiment comprises the use of two or more formulations herein in combination for stimulating hair growth.

### Embodiments List

The following list of embodiments does not limit the embodiments otherwise described herein.

1. A composition comprising, consisting essentially of, or consisting of hyaluronic acid and a medicament.

2. The composition of embodiment 1, where the hyaluronic acid is conjugated with the medicament via ester bonds.

3. The composition of any one or more of embodiments 1 to 2, where the ester bond are made by reacting acrylate modified hyaluronic acid with a chemical linker or with the medicament.

4. The composition of any one or more of embodiments 1 to 3, where the extent of linking is less than 5 percent.

5. The composition of any one or more of embodiments 1 to 4, where the extent of linking is between 0.01 to 5.0 percent.

6. The composition of any one or more of embodiments 1 to 5, where the extent of linking is at or between any two of 0.01, 0.05, 0.1, 0.5, 1.0, 2.0, 3.0, 4.0, or 5.0 percent.

7. The composition of any one or more of embodiments 1 to 6, where the acrylate of the acrylate modified hyaluronic acid reacts with a thiol of the medicament or linker.

8. A composition comprising, consisting essentially of, or consisting of a hyaluronic acid hydrogel, wherein the hyaluronic acid hydrogel crosslinks include crosslinked hydrolytically degradable bonds.

9. The composition of embodiment 8, where the extent of crosslinking is less than 5 percent.

10. The composition of any one or more of embodiments 8 to 9, where the extent of crosslinking is between 0.01 to 5.0 percent.

11. The composition of any one or more of embodiments 8 to 10, where the extent of crosslinking is at or between any two of 0.1, 0.5, 1.0, 2.0, 3.0, 4.0 or 5.0 percent.

12. The composition of any one or more of embodiments 8 to 11, where the hydrolytically degradable bonds comprise ester bonds.

13. The composition of any one or more of embodiments 8 to 12, where the hyaluronic acid hydrogel is made by crosslinking acrylate modified hyaluronic acid.

14. The composition of any one or more of embodiments 8 to 13, where the acrylate modified hyaluronic acid is crosslinked with multifunctional thiol molecules.

15. The composition of any one or more of embodiments 8 to 14, where the hyaluronic acid hydrogel forms microparticles in the range of 1 micron to 2 millimeter in diameter when reconstituted in aqueous buffer.

16. The composition of any one or more of embodiments 8 to 15, where the hyaluronic acid hydrogel forms microparticles between 100 and 500 microns.

17. The composition of any one or more of embodiments 8 to 16, where the hyaluronic acid hydrogel forms microparticles when reconstituted at 20 mg/mL in phosphate buffered saline, pH 7.4, at room temperature.

18. The composition of any one or more of embodiments 8 to 17, where the hyaluronic acid hydrogel has a tan delta less than 1 at 0.5 Hz.

19. The composition of any one or more of embodiments 8 to 18, where the hyaluronic acid hydrogel has a tan delta less than 1 at 2.5 Hz

20. The composition of any one or more of embodiments 8 to 19, where the hyaluronic acid hydrogel has a storage modulus in the range of 100 Pa to 10 kPa when reconstituted in aqueous buffer at 2 wt %.

21. The composition of any one or more of embodiments 8 to 20, where the hyaluronic acid hydrogel has a storage modulus in the range of 100 Pa to 10 kPa when reconstituted at 20 mg/mL in phosphate buffered saline, pH 7.4, at room temperature.

22. The composition of any one or more of embodiments 8 to 21 further comprising a medicament.

23. The composition of any one or more of embodiments 8 to 22, where the medicament is linked to the hyaluronic acid hydrogel.

24. The composition of any one or more of embodiments 8 to 23, where the medicament is the crosslinker of the hyaluronic acid hydrogel.

25. A method of treatment comprising, consisting essentially of, or consisting administering into a body of a subject the compositions of any one of embodiments 1 to 25.

26. The method of embodiment 25, where the treatment is for at least one of providing lubrication, treating inflammation, treating osteoarthritis, treating synovitis, treating ocular disease, treating cardiovascular disease, treating disc degeneration, treating diabetic ulcers, treating pain, reducing inflammation following surgery, or improving tissue healing following surgery or a traumatic injury.

27. The method of any one or more of embodiments 25 to 26, where the subject is a mammal.

28. The method of any one or more of embodiments 25 to 27, where the mammal is a human.

29. The method of any one or more of embodiments 25 to 27, where the mammal is a horse.

30. The method of any one or more of embodiments 25 to 27, where the mammal is a dog.

31. The method of any one or more of embodiments 25 to 30, where the treatment is for at least one of treating inflammation, treating osteoarthritis, treating synovitis, treating ocular disease, treating cardiovascular disease, treating disc degeneration, treating diabetic ulcers, treating pain, reducing inflammation following surgery, or improving tissue healing following surgery or a traumatic injury.

32. The method of any one or more of embodiments 25 to 31, where the administering into a body of the subject comprises injecting into pericardial fluid of the subject to treat heart disease.

33. The method of any one or more of embodiments 25 to 31, where the administering into a body of the subject comprises injecting into synovial fluid of the subject to treat synovitis or osteoarthritis.

34. The method of any one or more of embodiments 25 to 33, where hydrolytically degradable hyaluronic acid hydrogel microparticles are injected into the synovial fluid of the subject to treat osteoarthritis or synovitis.

35. The method of any one or more of embodiments 25 to 31 and 34, where the hydrolytically degradable hyaluronic acid hydrogel microparticles have a storage modulus of 100 Pa to 10 kPa.

36. The method of any one or more of embodiments 25 to 31 and 34 to 35, where the hydrolytically degradable hyaluronic acid hydrogel microparticles are at 10 to 40 mg/mL in aqueous buffer.

37. The method of any one or more of embodiments 25 to 31 and 34 to 36, where the hydrolytically degradable hyaluronic acid hydrogel microparticles are injected at 10 to 100% v/v of the subject's synovial fluid volume.

38. A composition comprising, consisting essentially of, or consisting of a hydrogel network and linked medicaments or drugs.

39. The composition of embodiment 38, where crosslinks of the hydrogel and drug linkages include ester bonds.

40. The composition of any one or more of embodiments 38 to 39, where the crosslinks and the links of the linked medicaments or drugs are formed through reaction of an acrylate and thiol.

41. The composition of any one or more of embodiments 38 to 40, where the hydrogel network comprises a polysaccharide chemically modified with acrylate groups.

### EXAMPLES

### Example 1

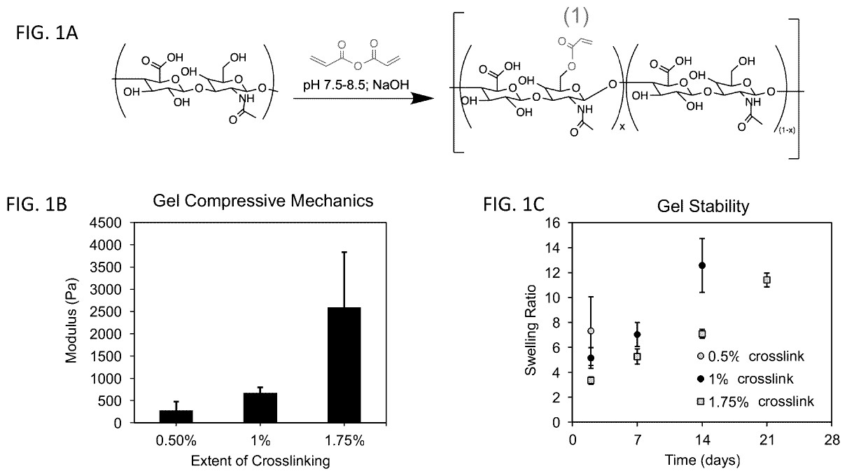

Sodium hyaluronate (700 kDa, Lifecore Biomedical) was dissolved in water at 1 g per 200 mL. 100, 60 or 30 microliters of acrylic anhydride (Sigma) was added with vigorous mixing and the pH was maintained between 7.0 and 9.0 for 18 hrs using automated titration equipment with 0.5M NaOH. After 18 hrs the acrylate modified HA was precipitated by adding 8% v/v 3M NaCl followed by 300% reagent alcohol. The acrylate modified HA was washed with 4:1 alcohol to water, followed by alcohol and dried under vacuum. Extent of acrylate modification was approximated at 1.75, 1 or 0.5 percent by extrapolating modification values of greater than 2 percent (measured with 1H NMR (Bruker 400 MHz)) based on molar ratios of acrylic anhydride to HA repeat unit under the same automated synthesis parameters. Purified acrylate modified hyaluronic acids were dissolved in phosphate buffer saline, pH 7.4 at 20 mg/mL and dithiothreitol was added for 1.75, 1 and 0.5% extent of crosslinking (1:1 thiol: theoretical acrylate ratio). The solutions were incubated at room temperature for 10 days and then subjected to compression testing. Compressive testing data is shown in FIG. 1B. 100 microliter hydrogels were incubated in PBS at 37 C and swelling ratios were calculated by subtracting the original mass from the measured mass and dividing by the original mass (FIG. 1C).

### Example 2

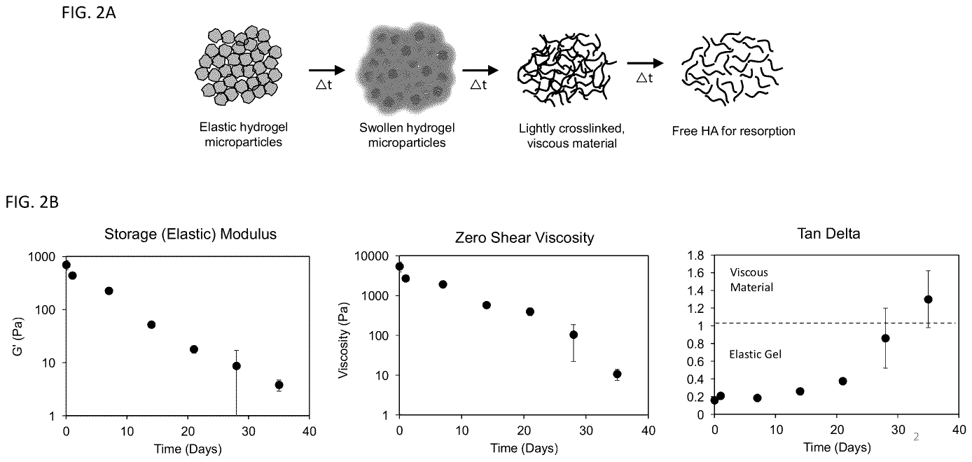

Sodium hyaluronate (700 kDa, Lifecore Biomedical) was dissolved in water at 1 g per 200 mL. 60 microliters of acrylic anhydride (Sigma) was added with vigorous mixing and the pH was maintained between 7.0 and 9.0 for 18 hrs using automated titration equipment with 0.5M NaOH. After 18 hrs the acrylate modified HA was precipitated by adding 8% v/v 3M NaCl followed by 300% reagent alcohol. The acrylate modified HA was washed with 4:1 alcohol to water, followed by alcohol and dried under vacuum. Percent acrylate modification was approximated at 1 percent by extrapolating modification values of greater than 2 percent (measured with 1H NMR (Bruker 400 MHz)) based on molar ratios of acrylic anhydride to HA repeat unit under the same automated synthesis parameters. Purified acrylate modified hyaluronic acid was dissolved in phosphate buffer saline, pH 7.4 at 20 mg/mL and dithiothreitol was added for 1% extent of crosslinking (1:1 thiol: theoretical acrylate). The solutions were incubated at room temperature for 7 days and then fragmented into microparticles by passing through screens with 720 and 330 mesh sizes, swollen in 400% (w/w) water, then passed through screens with 110 micron mesh size. The microparticles were then then precipitated with reagent alcohol, washed with a 4:1 reagent alcohol to water mixture, washed with reagent alcohol and then vacuum dried to form a powder. The microparticle powder was reconstituted with phosphate buffered saline, pH 7.4 at 20 mg/mL and then 100 milligrams was added to the upper well of a transwell insert (8 micron pore diameter) and incubated against phosphate buffered saline, pH 7.4 at 37° C. Rheological properties of the microparticles in the transwells were measured weekly using oscillatory rheology (1 degree cone and plate, 0.5 Hz, TA instruments). Rheological data is shown in FIG. 2B.

### Example 3

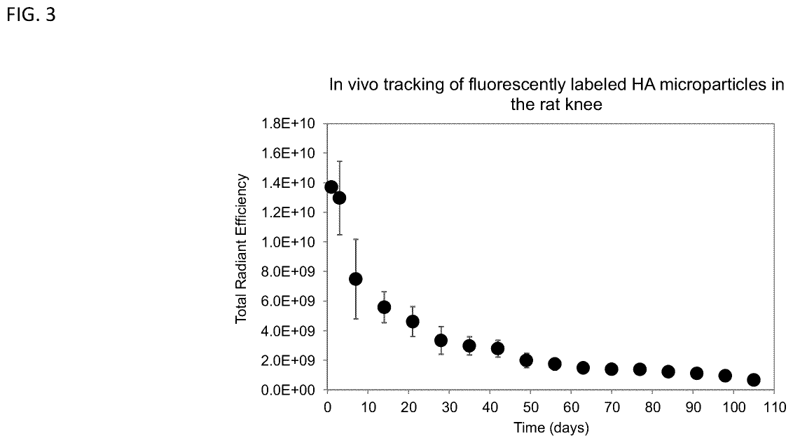

Sodium hyaluronate (700 kDa, Lifecore Biomedical) was dissolved in water at 0.3 g per 300 mL MES buffered saline, pH 4.7 (Thermo Scientific). 50 mg of cyanine 7.5 amine (Lumiprobe) was dissolved in 5 mL dimethylsulfoxide then 5 ml of water was added on ice with mixing. The cyanine 7.5 amine solution was dripped into the sodium hyaluronate solution with mixing. 200 mg of (4-(4,6-Dimethoxy-1,3,5-triazin-2-yl)-4-methylmorpholinium Chloride) (DMTMM, TCl) was dissolved in 10 ml of water and dripped into sodium hyaluronate solution. The solution was mixed at room temperature for 23 hrs then 18 mL of a 3M sodium chloride solution was dripped in with mixing. The sodium hyaluronate was precipitated by dripping in 0.75 L of reagent alcohol (Fisher Scientific) with mixing and pelleted with centrifugation. The pellet was redissolved in 300 ml water, then precipitated again with 18 mL 3M sodium chloride and 0.75 L reagent alcohol and pelleted with centrifugation. The pellet was redissolved in 300 ml water, then precipitated one more time with 18 mL 3M sodium chloride and 0.75 L reagent alcohol and pelleted with centrifugation. The pellet was washed with 1L of a 4:1 water: reagent alcohol mixture, and then 250 mL of reagent alcohol before drying under vacuum for 6 hrs. The cyanine 7.5 labeled hyaluronate was dissolved at 0.1 g in 20 mL water then 17 microlites of acrylic anhydride was added with mixing. The mixture was maintained between pH 7.0 and pH 9.0 with automated synthesis equipment for 17 hrs. The cyanine 7.5 labeled and acrylate modified hyaluronic acid was precipitated by adding 1.5 mL 3M sodium chloride followed by 70 mL reagent alcohol with mixing. The precipitate was pelleted with centrifugation and then washed with 45 mL of a 4:1 reagent alcohol to water mixture, followed by 45 mL of reagent alcohol and then pelleted by centrifugation and dried under vacuum for three hours. The cyanine 7.5 labeled and acrylate modified hyaluronic acid was dissolved in phosphate buffered saline, pH 7.4 at 66 mg in 3 mL for 2 hrs with intermittent mixing. 0.33 mL of a 1.14 mg/mL dithiothreitol was added to the cyanine 7.5 labeled and acrylate modified hyaluronic acid and mixed uniformly. The solution was incubated for 7 days at room temperature. The resultant hydrogel was fragmented by passing through 770 and 330 micron screens 5 times each. 25 mL water was added to the hydrogel particles and mixed for 0.5 hr at 22 degrees C. then passed through a 110 micron screen. The particles were then precipitated by adding 1.8 mL 3M sodium chloride followed by 70 mL reagent alcohol. The precipitate was pelleted, washed with 50 mL 4:1 reagent alcohol: water followed by 50 mL reagent alcohol, pelleted and vacuum dried for two hours. The resultant particles were resuspended in phosphate buffered saline, pH 7.4 at 20 mg/mL and rheological properties were measured (1 degree cone and plate, 0.5 Hz, TA Instruments, Table 1). 25+/−2 mg of the cyanine 7.5 labeled hyaluronic acid hydrogel particles were loaded into 31 G insulin syringes for administration into rats. Male Lewis rats (250-275 g) were anesthetized with isoflurane, knees shaved with clippers and hyaluronic acid hydrogel particles administered via intra-articular injection through a 31G needle according to approved IACUC protocol. The fluorescent signal of the near-IR cyanine 7.5 label was measured over time with serial imaging of the rat knee using an IVIS Lumina (780/845 nm excitation/emission, FIG. 3, n=6 measurements at each timepoint, mean±/−standard deviation).

TABLE 1
Rheological properties of cyanine 7.5
labeled hyaluronic acid hydrogel particles.

| | | |
|---|---|---|
| G′@0.5 Hz | 524 | 582 |
| G″@0.5 Hz | 44.3 | 54.3 |
| Tan delta | 0.08 | 0.09 |
| 0.5 Hz | | |
| G′ @2.5 Hz | 574 | 639 |
| G″ @2.5 Hz | 57.6 | 67.6 |
| Tan delta | 0.10 | 0.11 |
| 2.5 Hz | | |
| N0 | 18208 | 13710 |
| N0.1 | 2968 | 2364 |
| N1 | 303 | 263 |
| N10 | 35.3 | 33.1 |
| N100 | 6.1 | 7.2 |

### Example 4

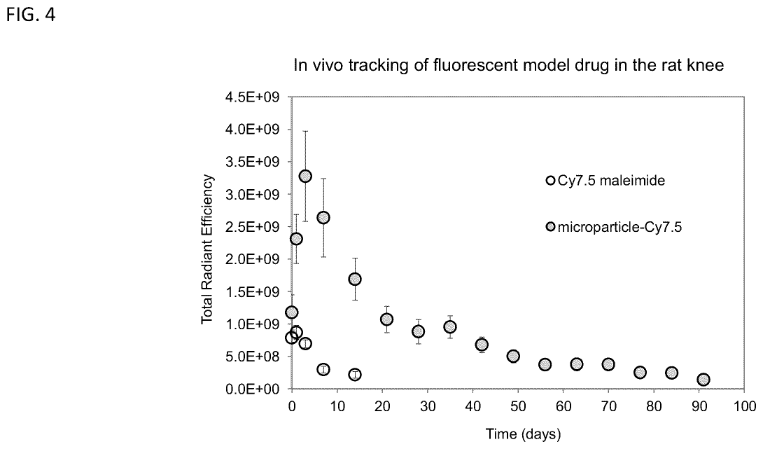

Sodium hyaluronate (700 kDa, Lifecore Biomedical) was dissolved in water at 1 g per 200 mL. 100 microliters of acrylic anhydride (Sigma) was added with vigorous mixing and the pH was maintained between 7.0 and 9.0 for 18 hrs using automated titration equipment with 0.5M NaOH. After 18 hrs the acrylate modified HA was precipitated by adding 8% v/v 3M NaCl followed by 300% reagent alcohol. The acrylate modified HA was washed with 4:1 alcohol to water, followed by alcohol and dried under vacuum. Percent acrylate modification was approximated at 1.75 percent by extrapolating modification values of greater than 2 percent (measured with 1H NMR (Bruker 400 MHz)) based on molar ratios of acrylic anhydride to HA under the same automated synthesis parameters. Purified acrylate modified hyaluronic acid was dissolved in phosphate buffer saline at 20 mg/mL and dithiothreitol was added for 1.75% extent of crosslinking (1:1 thiol: theoretical acrylate). The solutions were incubated at room temperature for 7 days and then fragmented into microparticles by passing through screens with 720 and 330 mesh sizes, swollen in 400% (w/w) water, then passed through screens with 110 micron mesh size. The microparticles were then then precipitated with reagent alcohol, washed with a 4:1 reagent alcohol to water mixture, washed with reagent alcohol and then vacuum dried to form a powder. Rheological properties of hydrogel microparticles reconstituted with phosphate buffered saline, pH 7.4 at 20 mg/ml are shown in Table 2. Cyanine-7.5 maleimide (Lumiprobe) was linked to the crosslinked hyaluronic acid acrylate microparticles by reconstituting the microparticle powder in an aqueous buffer, pH 7.4 with cyanine-7.5 maleimide at a concentration of 0.25 micrograms per milliliter. The reconstituted hydrogel microparticles were incubated for 1 hour at room temperature prior to intra-articular administration in rats. 25+/−2 mg of the cyanine 7.5 linked hyaluronic acid hydrogel particles were loaded into 31 G insulin syringes for administration into rats. 25+/−3 mg of the 0.25 microgram per milliliter cyanine 7.5 maleimide dye solution was loaded into 31 G insulin syringes as a control. Male Lewis rats (250-275 g) were anesthetized with isoflurane, knees shaved with clippers and hyaluronic acid hydrogel particles or cyanine 7.5 maleimide control were administered via intra-articular injection through a 31G needle according to an approved IACUC protocol. The fluorescent signal of the near-IR cyanine 7.5 dye was measured over time with serial imaging of the rat knee using an IVIS Lumina (780/845 nm excitation/emission, FIG. 4, n=6 measurements at each timepoint, mean+/−standard deviation).

TABLE 2
Rheological properties of hyaluronic acid hydrogel
microparticles before linking cyanine 7.5 maleimide.
Measurements from two samples shown.

| | | |
|---|---|---|
| G′@0.5 Hz | 673 | 515 |
| G″@0.5 Hz | 139 | 107 |
| Tan delta | 0.21 | 0.21 |
| 0.5 Hz | | |
| G′ @2.5 Hz | 832 | 647 |
| G″ @2.5 Hz | 176 | 145 |
| Tan delta | 0.21 | 0.22 |
| 2.5 Hz | | |
| N0 | 6700 | 4899 |
| N0.1 | 1007 | 633 |
| N1 | 152 | 82.9 |
| N10 | 31.5 | 18.8 |
| N100 | 6.8 | 4.1 |

### Example 5

Hyaluronic acid hydrogel microparticles were made as in Example 2 and reconstituted at 20 mg/mL in phosphate buffered saline, pH 7.4. Rheological properties are shown in Table 3. Synovial fluid was collected from the tarsal and carpal joints of three healthy 2-year old horses (1 mare and 2 geldings, 3-6 mL collected from each joint) and then 2 mL of the reconstituted microparticles were injected through a 18G syringe (2 joints injected per horse. 2 joints injected with phosphate buffered saline per horse as control). Lameness assessments were performed prior to injections and then weekly for 4 weeks. Synovial fluid was collected and analyzed for cellular content prior to injections and then after 2 weeks and 4 weeks. No joint swelling, lameness or changes to synovial fluid cellular content compared to control joints were observed following microparticle administration.

TABLE 3
Rheological properties of hyaluronic acid hydrogel
microparticles administered to the horse.
Measurements from two samples shown.

| | | |
|---|---|---|
| G′@0.5 Hz | 386 | 444 |
| G″@0.5 Hz | 75.0 | 84.1 |
| Tan delta | 0.19 | 0.19 |
| 0.5 Hz | | |
| G′ @2.5 Hz | 496 | 537 |
| G″ @2.5 Hz | 123 | 132 |
| Tan delta | 0.25 | 0.25 |
| 2.5 Hz | | |
| N0 | 1958 | 2580 |
| N0.1 | 218 | 275 |
| N1 | 30.8 | 36.6 |
| N10 | 7.4 | 8.3 |
| N100 | 2.0 | 2.3 |

### Example 6

Sodium hyaluronate (700 kDa, Lifecore Biomedical) was dissolved in water at 1 g per 200 mL. 100 microliters of acrylic anhydride (Sigma) was added with vigorous mixing and the pH was maintained between 7.0 and 9.0 for 18 hrs using automated titration equipment with 0.5M NaOH. After 18 hrs the acrylate modified HA was precipitated by adding 8% v/v 3M NaCl followed by 300% reagent alcohol. The acrylate modified HA was washed with 4:1 alcohol to water, followed by alcohol and dried under vacuum. Percent acrylate modification was approximated at 1.75 percent by extrapolating modification values of greater than 2 percent (measured with 1H NMR (Bruker 400 MHz)) based on molar ratios of acrylic anhydride to HA under the same automated synthesis parameters. Purified acrylate modified hyaluronic acid was dissolved in phosphate buffer saline at 20 mg/mL and dithiothreitol was added for 1.75% extent of crosslinking (1:1 thiol: theoretical acrylate). The solutions were incubated at room temperature for 7 days and then fragmented into microparticles by passing through screens with 720 and 330 mesh sizes, swollen in 400% (w/w) water, then passed through screens with 110 micron mesh size. The microparticles were then precipitated with reagent alcohol, washed with a 4:1 reagent alcohol to water mixture, washed with reagent alcohol and then vacuum dried to form a powder. The microparticle powder was reconstituted with phosphate buffered saline, pH 7.4 at 20 mg/mL. Rheological properties are shown in Table 4. A 15-year old Labrador mix (male, 65 lbs) was previously diagnosed with hip dysplasia and severe osteoarthritis. 2 mL of the reconstituted microparticles were administered to each hip joint through an 18G needle under general anesthesia. No adverse events were observed and the dog resumed normal activity within 24 hrs. Improvements in the dog's ability to get up from his bed, his willingness to go on walks and his pace during walks were observed over the first month following treatment. The dog was unable to complete the 8-meter walk for quantified gait analysis prior to treatment due to hip pain and instability, but was able to at the 30 day follow-up evaluation.

TABLE 4
Rheological properties of hyaluronic acid hydrogel
microparticles administered to the dog.
Measurements from two samples shown.

| | | |
|---|---|---|
| G′@0.5 Hz | 673 | 515 |
| G″@0.5 Hz | 139 | 107 |
| Tan delta | 0.21 | 0.21 |
| 0.5 Hz | | |
| G′ @2.5 Hz | 832 | 647 |
| G″ @2.5 Hz | 176 | 145 |
| Tan delta | 0.21 | 0.22 |
| 2.5 Hz | | |
| N0 | 6700 | 4899 |
| N0.1 | 1007 | 633 |
| N1 | 152 | 82.9 |
| N10 | 31.5 | 18.8 |
| N100 | 6.8 | 4.1 |

### Example 7

Sodium hyaluronate (700 kDa, Lifecore Biomedical) was dissolved in water at 1 g per 200 mL. 100 microliters of acrylic anhydride (Sigma) was added with vigorous mixing and the pH was maintained between 7.0 and 9.0 for 18 hrs using automated titration equipment with 0.5M NaOH. After 18 hrs the acrylate modified HA was precipitated by adding 8% v/v 3M NaCl followed by 300% reagent alcohol. The acrylate modified HA was washed with 4:1 alcohol to water, followed by alcohol and dried under vacuum. Percent acrylate modification was approximated at 1.75 percent by extrapolating modification values of greater than 2 percent (measured with 1H NMR (Bruker 400 MHz)) based on molar ratios of acrylic anhydride to HA under the same automated synthesis parameters. Purified acrylate modified hyaluronic acid was dissolved in phosphate buffer saline at 20 mg/mL and dithiothreitol was added for 1% extent of crosslinking. The solutions were incubated at room temperature for 7 days and then fragmented into microparticles by passing through screens with 720 and 330 mesh sizes, swollen in 400% (w/w) water, then passed through screens with 110 micron mesh size. The microparticles were then precipitated with reagent alcohol, washed with a 4:1 reagent alcohol to water mixture, washed with reagent alcohol and then vacuum dried to form a powder. The microparticle powder was reconstituted with phosphate buffered saline, pH 7.4 at 20 mg/mL. Rheological properties are shown in Table 5. Microparticle powder was reconstituted at 20 mg in 2 mL of phosphate buffer saline and delivered to the pericardial cavity of pigs through a minimally invasive, catheter-based procedure. No changes in heart electrical signaling, contractility or hemodynamics were observed over 60 days.

TABLE 5
Rheological properties of hyaluronic acid hydrogel
microparticles administered to the pig.
Measurements from two samples shown.

| | | |
|---|---|---|
| G′@0.5 Hz | 346 | 329 |
| G″@0.5 Hz | 74.4 | 70.5 |
| Tan delta | 0.21 | 0.21 |
| 0.5 Hz | | |
| G′ @2.5 Hz | 429.2 | 407 |
| G″ @2.5 Hz | 96.9 | 89.8 |
| Tan delta | 0.23 | 0.22 |
| 2.5 Hz | | |
| N0 | 3463 | 5776 |
| N0.1 | 562 | 701 |
| N1 | 86.5 | 95.9 |
| N10 | 19.5 | 18.5 |
| N100 | 4.7 | 4.7 |

1. A composition comprising hyaluronic acid and a medicament, wherein the hyaluronic acid is conjugated with the medicament via ester bonds, and the composition is made by reacting acrylate modified hyaluronic acid with a chemical linker or the medicament.

2. The composition of claim 1, wherein the extent of acrylate modification of the hyaluronic acid is less than 5 percent, preferably between 0.01 to 5.0 percent, and more preferably at or between any two of 0.01, 0.05, 0.1, 0.5, 1.0, 2.0, 3.0, 4.0, or 5.0 percent.

3. The composition of claim 2, wherein the acrylate of the acrylate modified hyaluronic acid reacts with a thiol of the medicament or a thiol of the chemical linker.

4. A composition comprising a hyaluronic acid hydrogel, wherein crosslinks of the hyaluronic acid hydrogel include ester bonds, wherein the extent of crosslinking of the hyaluronic acid hydrogel is less than 5 percent, preferably between 0.01 to 5.0 percent, and more preferably at or between any two of 0.1, 0.5, 1.0, 2.0, 3.0, 4.0 or 5.0 percent.

5. (canceled)

6. The composition of claim 4, wherein the hyaluronic acid hydrogel is made by crosslinking acrylate modified hyaluronic acid.

7. The composition of claim 6, wherein the acrylate modified hyaluronic acid is crosslinked with multifunctional thiol molecules.

8. The composition of claim 4, wherein the hyaluronic acid hydrogel forms microparticles in the range of 1 micron to 2 millimeter in diameter, preferably between 100 and 500 microns, when reconstituted at 20 mg/mL in phosphate buffered saline, pH 7.4, room temperature.

9-10. (canceled)

11. The composition of claim 4, wherein the hyaluronic acid hydrogel has a storage modulus in the range of 100 Pa to 10 kPa, preferably between 100 Pa and 1k Pa via oscillatory rheology, 1 degree cone and plate, 2.5 Hz, when reconstituted at 20 mg/mL in phosphate buffered saline, pH 7.4, room temperature.

12. The composition of claim 4, further comprising a medicament, wherein the medicament is linked to the hyaluronic acid hydrogel.

13. (canceled)

14. A method of treatment comprising administering into a body of a subject the composition of claim 4, wherein the treatment is for at least one of providing lubrication, treating inflammation, treating osteoarthritis, treating synovitis, treating ocular disease, treating cardiovascular disease, treating disc degeneration, treating diabetic ulcers, treating pain, reducing inflammation following surgery, or improving tissue healing following surgery or a traumatic injury.

15-18. (canceled)

19. The method of claim 14, wherein the administering into a body of the subject comprises injecting into pericardial fluid of the subject to treat heart disease.

20. The method of claim 14, wherein the administering into a body of the subject comprises injecting into synovial fluid of the subject to treat synovitis or osteoarthritis.

21-22. (canceled)

23. A composition comprising a hydrogel network and linked medicaments or drugs, wherein hydrogel crosslinks and medicament or drug linkages include ester bonds.

24. The composition of claim 23, wherein the crosslinks and the linkages are formed through reaction of an acrylate and thiol.

25. The composition of claim 24, wherein the hydrogel network comprises a polysaccharide chemically modified with acrylate groups.
